(19) United States
(12) Patent Application Publication
(10) Pub. No.: US 2007/0272620 A1
(43) Pub. Date: Nov. 29, 2007

(54) COMBINED FEED TUBE ADAPTER AND SANITIZER UNIT FOR A BOTTLED WATER COOLER

(75) Inventor: David B. Chaney, Westerville, OH (US)

Correspondence Address: KELLY LOWRY & KELLEY, LLP, 6320 CANOGA AVENUE, SUITE 1650, WOODLAND HILLS, CA, 91367, US

(73) Assignee: ZOHAR WATERWORKS, LLC, Charlotte, NC (US)

(21) Appl. No.: 11/745,026

(22) Filed: May 7, 2007

Related U.S. Application Data
(60) Provisional application No. 60/767,532, filed on May 23, 2006.

(51) Int. Cl. C02F 1/32

(52) U.S. Cl. 210/748

(57) ABSTRACT

A combined feed tube mounting adapter and sanitizer unit for use in a bottled water cooler having a cooler reservoir for receiving water from a bottle mounted in an inverted orientation for water downflow from the bottle to the reservoir. The combined unit includes an adapter body removably mounted over the cooler reservoir, with an upstanding feed tube or probe for operatively engaging a valved bottle cap on the neck of the inverted water-containing bottle. An air filter is mounted on the adapter body along an air inflow path to filter ambient air drawn into the reservoir. The combined unit further includes a flow line for introducing ozone gas into the reservoir-contained water. In a preferred form, this flow line is coupled with a gas generator which is operated by a controller mounted in a user-accessible position on the adapter body.

### BACKGROUND OF THE INVENTION

This invention relates generally to improvements in bottled water coolers particularly of the type having a feed tube adapter equipped with a feed tube or probe for operating a valved bottle cap carried on the neck of a water-containing bottle to open the bottle cap incident to bottle mounting in an inverted orientation onto the water cooler, so that water can flow downwardly from the bottle into a cooler reservoir. More particularly, this invention relates to an improved feed tube adapter incorporating means for introducing a sanitizing gas such as ozone into the cooler reservoir to maintain water therein in a clean and healthy state.

Bottled water coolers are generally known in the art to comprise a cooler housing supporting an upwardly open reservoir to receive a supply of water for on-demand dispensing via one or more faucet valves or the like. The cooler housing and/or the reservoir are designed to support a water bottle of typically three to five gallon capacity in an inverted orientation over the reservoir, so that water contained within the bottle may flow downwardly into and fill the reservoir to a level sufficient to cover an open bottle mouth formed in a bottle neck. In this regard, downward water flow from the bottle is accompanied by an upward exchange of air passing from the reservoir into the bottle to replace the volume of water displaced or discharged from the bottle. This air-water exchange between the overlying bottle and the underlying reservoir continues until the reservoir water level rises sufficiently to cover the bottle mouth, at which time upward air exchange is halted to correspondingly stop downward water flow. Subsequent dispensing of water from the reservoir by operation of the faucet valves causes the water level within the reservoir to fall below and thus uncover the bottle mouth, whereupon the air-water exchange may resume to enable additional water to flow downwardly from the bottle to refill the reservoir. In many modern bottled water coolers of this general type, the reservoir may be associated with refrigeration means for chilling at least a portion of the water contained therein to provide chilled water used primarily for drinking or for use in making chilled beverages.

In the past, it has been conventional to provide the water bottle to a customer in a substantially filled state with the bottle mouth closed and sealed by a bottle cap. This bottle cap has normally included a tear-away skirt to allow the customer to remove the cap immediately prior to installation of the bottle in an inverted orientation on the cooler. More recently, alternative valved bottle caps have been designed to remain on the water bottle when the bottle is installed onto the cooler, wherein such alternative bottle caps are designed to be engaged and opened by a feed tube or probe mounted on the cooler in a position over the reservoir. See, for example, U.S. Pat. Nos. 4,874,023; 5,222,531; 5,232,125; 5,413,152; 6,167,921; and 6,619,511. In these arrangements, the feed tube or probe is provided as a portion of a mounting adapter installed onto the cooler to extend over and substantially cover the top of the reservoir to reduce or prevent entry of dirt and other contaminants. The feed tube or probe has a contoured head or tip for engaging a valve plug on the bottle cap to open a flow path as an incident to bottle installation, thereby permitting bottle installation onto the cooler with little or no water spillage. The feed tube or probe may further be designed to return the valve plug to a closed position on the cap as the bottle is removed from the cooler.

Feed tube adapters of this general type are normally equipped with a seal ring gasket or the like for sealingly engaging the reservoir, and an air filter is mounted on the adapter to filter air drawn into the cooler reservoir along an entry or air inflow path. With this construction, the water within the reservoir is protected against contact with airborne particulate and contaminants which may be present in ambient air. In many adapter designs, the seal ring gasket is carried by the mounting adapter in a position to bindingly engage an interior wall segment of the cooler reservoir with a sufficient engagement force whereby the gasket additionally performs the function of retaining the adapter in position on the reservoir during normal use.

Despite substantial commercialization of such feed tube adapters, there has remained an on-going concern directed to potential contamination of water contained within the cooler reservoir by biological organisms particularly such as bacterial organisms. In this regard, sanitizing systems have been proposed wherein a sanitizing agent such as ozone gas is periodically injected into the reservoir-contained water to safeguard against growth of undesirable and potentially unhealthy micro-organisms. See, e.g., U.S. Pat. Nos. 6,289,690; and 6,561,382; and U.S. Publication 2003/0000966. In the past, such sanitizing systems have required various gas flow lines to be coupled with the cooler reservoir as by connection through holes formed in reservoir side walls, whereby such concepts have not been suitable for quick and easy retrofit of water coolers currently in use. Moreover, such sanitizing systems have not been designed for compatibility with the above-described feed tube adapters.

The present invention provides an improved construction for a feed tube mounting adapter, wherein the feed tube adapter comprises a portion of a combined unit including means for introducing a sanitizing agent such as ozone gas into the water contained within the reservoir of a bottled water cooler. This combined unit is designed for quick and easy in-the-field retrofit installation onto a water cooler.

### SUMMARY OF THE INVENTION

In accordance with the invention, an improved and combined feed tube mounting adapter and sanitizer unit is provided for use in or on a bottled water cooler of the type having a cooler reservoir for receiving water from a bottle mounted on the cooler in an inverted orientation for water downflow from the bottle to the reservoir. The combined unit includes an adapter body removably mounted over the cooler reservoir, with an upstanding feed tube or probe for operatively engaging a valved bottle cap on the neck of the inverted water-containing bottle. The combined unit further includes a flow line for introducing a sanitizing agent such as ozone gas into the reservoir-contained water.

In a preferred form, the combined adapter and sanitizing unit comprises the adapter body for removably mounting onto and/or within an upper region of the cooler reservoir in substantially sealed engagement therewith. The upstanding feed tube or probe on the adapter body engages the valved bottle cap on the inverted water-containing bottle for opening a flow path incident to bottle mounting or placement onto the water cooler, to permit water downflow from the bottle and into the underlying reservoir. One or more dispense faucets on the water cooler permit on-demand dispensing of water from the cooler reservoir. As water is dispensed from the reservoir, the reservoir is re-filled or maintained in a substantially filled condition by means of additional water downflow from the inverted water bottle. An air filter is mounted on the adapter body along an air inflow path to filter ambient air drawn into the reservoir. Such adapter body and feed tube are constructed generally according to U.S. Pat. Nos. 5,413,152; 6,167,921; and 6,619,511, which are incorporated by reference herein.

The adapter body additionally accommodates a flow line coupled between a sanitizing agent source, such as an ozone gas generator positioned outside the cooler reservoir, and a delivery element mounted within the cooler reservoir. In the preferred form, the delivery element comprises a porous member such as a porous stone mounted at a downstream end of the flow line and positioned within a lower region of the reservoir. Ozone gas or the like delivered from the source through the flow line is thus converted by the porous member into a myriad of small gas bubbles which are delivered to a lower region and bubbled upwardly through the reservoir-contained water for sanitizing the reservoir interior and the water contained therein.

In accordance with a further aspect of the invention, a controller is mounted on the adapter body in a user-accessible position, such as at a forwardly presented position, and is adapted for user manipulation to select and/or program a periodic operating cycle for the sanitizing agent source. This controller conveniently comprises a digital display in association with control buttons to permit user-controlled selection of a cycle time and cycle duration for source operation, such as operating the source on a daily basis for a 10 minute cycle beginning at 2:00 a.m. With this arrangement, regular sanitization of the reservoir-contained water may take place, to insure water cleanliness of water within the reservoir, without interfering with normal day-time dispensing and use of the water.

When the sanitizing agent such as ozone gas is delivered to the water-containing reservoir, gas bubbled upwardly through the water is allowed to escape through the air inflow path and associated air filter thereby preventing undesired pressurization of the reservoir. In this regard, the air filter may incorporate a float-activated check valve of the type disclosed in U.S. Pat. No. 6,167,921 to preclude water outflow via the air inflow path in the event of reservoir over-filling, e.g., due to a cracked bottle or the like. However, such float-activated check valve will not preclude escape of the gas sanitizing agent to prevent reservoir pressurization.

Other features and advantages of the invention will become more apparent from the following detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a fragmented front perspective view illustrating an upper portion of a bottled water cooler with an inverted water bottle mounted thereon, wherein the cooler is equipped with an improved combined feed tube adapter and sanitizer unit, such as an ozonator unit, embodying the novel features of the invention;

FIG. 2 is a fragmented front perspective view of the bottled water cooler depicted in FIG. 1, with an inverted water bottle removed therefrom;

FIG. 3 is an enlarged front perspective view of the improved feed tube adapter unit;

FIG. 4 is a fragmented rear perspective view of the bottled water cooler, and depicting the improved feed tube adapter unit mounted thereon, with portions of a cooler housing and a water reservoir shown in shadow lines to illustrate construction details of the adapter unit;

FIG. 5 is a rear perspective view of the feed tube adapter unit shown removed from the water cooler; and

FIG. 6 is an enlarged vertical sectional view showing the feed tube adapter unit mounted onto the water cooler in functional relation with the water reservoir therein.

### DETAILED DESCRIPTION OF THE PREFERRED EMBODIMENTS

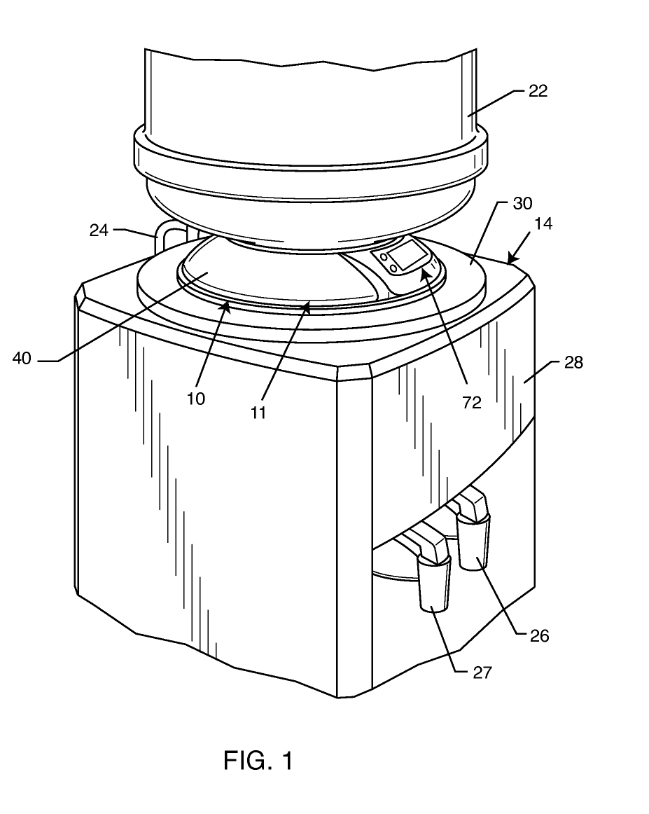

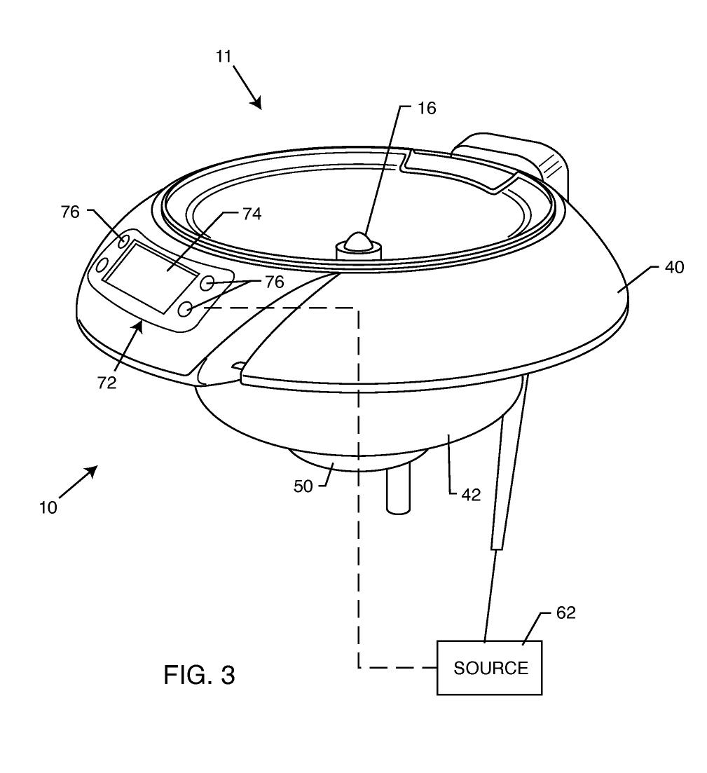

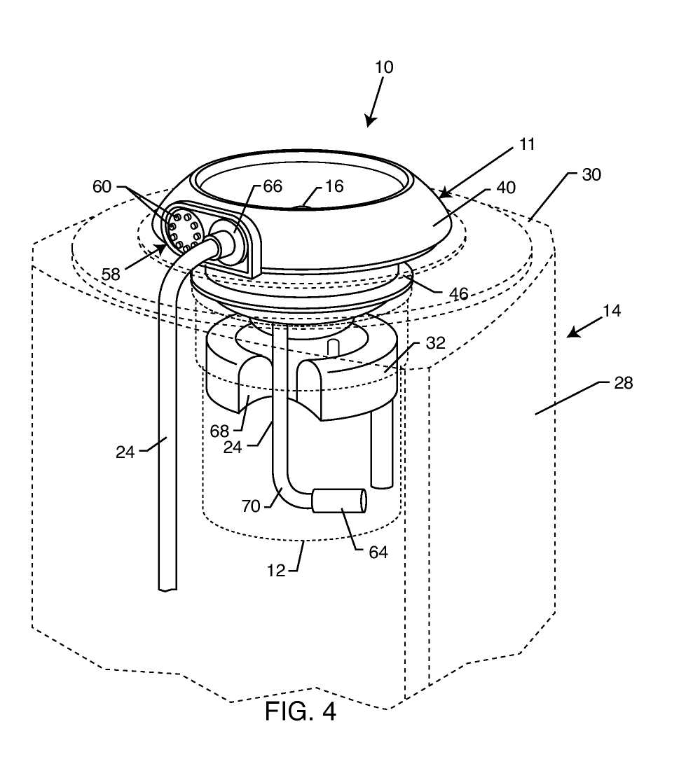

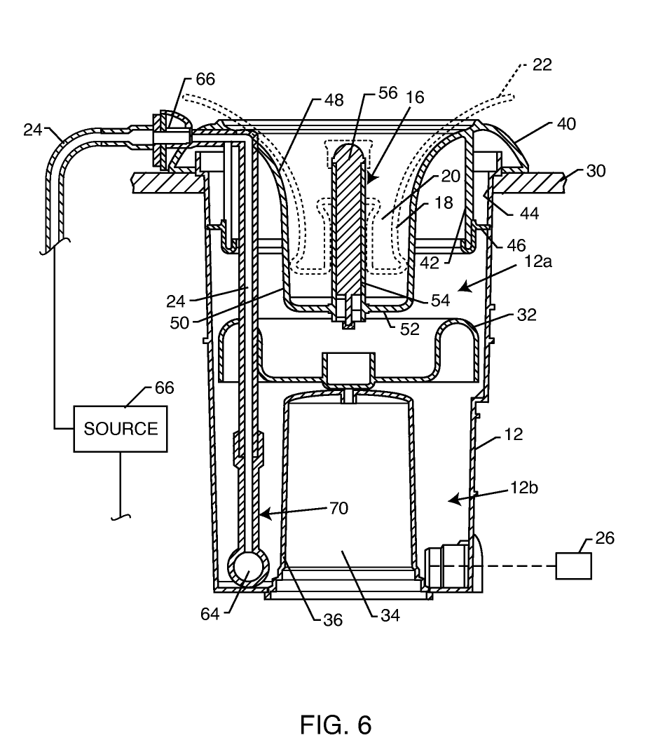

As shown in the exemplary drawings, a combined feed tube adapter and sanitizer unit referred to generally by the reference numeral 10 in FIGS. 1-6 is provided for removable installation onto or over the reservoir 12 (FIGS. 4 and 6) of a bottled water cooler 14 (FIGS. 1-2 and 4). The combined unit 10 comprises a mounting adapter 11 (shown best in FIG. 3) having an upstanding feed tube or probe 16 for engaging and opening a valved bottle cap 18 (FIG. 6) mounted on the neck 20 of an inverted water-containing bottle 22, to permit water downflow from the bottle 22 into the cooler reservoir 12. In addition, the combined unit 10 carries a flow line 24 for periodic inflow of a sanitizing agent such as ozone gas to maintain the reservoir interior and reservoir-contained water in a clean and sanitary state.

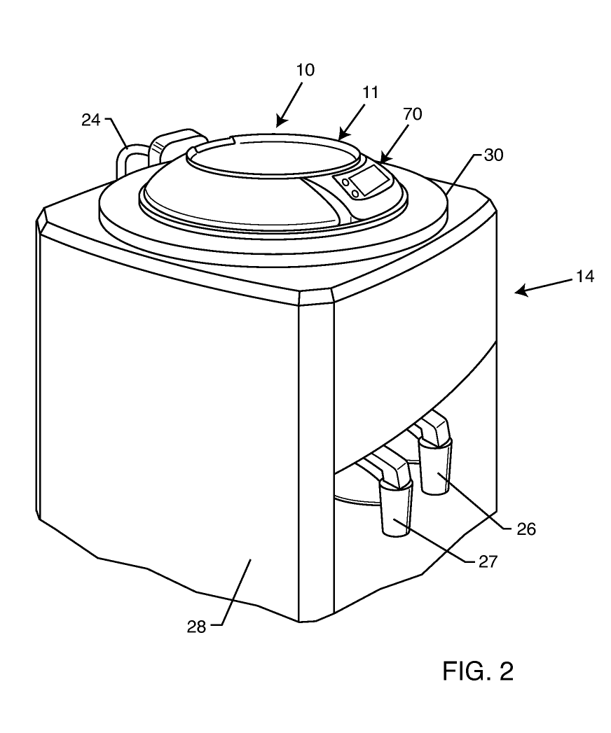

The combined feed tube adapter and ozonator unit 10 of the present invention is designed for use with a bottled water cooler 14 of the type having an upwardly open reservoir 12 for receiving and storing a supply of water for ready dispensing upon operation of one or more dispense or faucet valves 26, 27 or the like. In this regard, the water cooler 14 shown generally in FIGS. 1 and 2 typically comprises a cooler housing or cabinet 28 with the reservoir 12 (FIGS. 4 and 6) mounted within an upper region thereof and defining an upper end exposed through a cabinet top or lid 30. A water bottle 22 of typically five gallon capacity is mounted onto the cooler 14 in an inverted orientation at the top 30 of the cabinet 28 so that water within the bottle 22 can flow downwardly into and fill the underlying reservoir 12. In one typical water cooler design, the reservoir 12 is designed for removable mounting within the cabinet 28, and the interior of the reservoir 12 is subdivided (FIG. 6) by an internal baffle plate 32 into an upper chamber 12a for containing water substantially at room temperature, and a lower chamber 12b in thermal association with a chiller probe 34 which may slide-fit into a downwardly open chiller sleeve 36 formed in a bottom wall of the reservoir 12. Separate faucet valves 26 and 27 are mounted on the face of the cooler cabinet 28 (FIGS. 1 and 2) for separately dispensing water from these different-temperature compartments 12a and 12b. In this regard, the general construction and operation of the illustrative bottled water cooler 14 is described in more detail in U.S. Pat. Nos. 5,246,141; 5,307,958; 5,289,951; 5,395,014; 5,297,700; and 6,167,921 which are incorporated by reference herein.

The combined unit 10 generally comprises the mounting adapter 11 in the form of a compact adapter body or shell having a relatively simple and preferably plastic molded construction. More particularly, the illustrative adapter body 16 includes an upper support rim 40 projecting radially outwardly a short distance from the upper margin of a generally cylindrical sleeve segment 42 (shown best in FIG. 6), wherein the support rim 40 is sized and shaped to rest upon the cabinet lid or top 30 with the sleeve segment 42 extending downwardly from the support rim 40 through an opening 44 in the cabinet top 30, and further at least partially into an upper region of the cooler reservoir 14. An annular seal gasket 46 is carried by the sleeve segment 42 for bindingly and sealingly engaging an interior wall surface of the reservoir 14.

In addition, the adapter 11 includes an inner funnel segment 48 extending radially inwardly from the support rim 40 and curving smoothly in a downward direction to merge with an upwardly open inner cup segment 50 having the feed tube or probe 16 upstanding centrally from a bottom or base wall 52 thereof. This feed tube or probe 16, as previously described, is configured to engage and open the valved bottle cap 18 on the inverted water bottle 22 incident to bottle placement of mounting onto the water cooler 14. FIG. 6 shows the feed tube or probe 16 in the form of a tubular post 54 having a feed tube insert 56 removably mounted therein as by means of snap-fit assembly, as shown and described in U.S. Pat. No. 6,619,511 which is incorporated by reference herein. Persons skilled in the art will appreciate that the adapter 11 with associated feed tube or probe 16 may take a variety of alternative constructions known in the art.

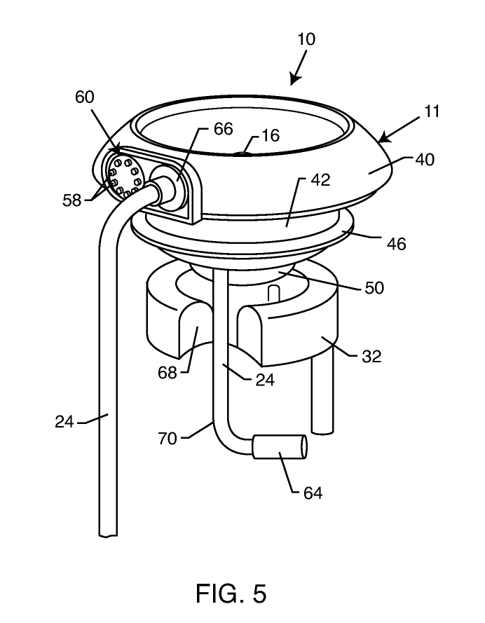

The adapter 11 is further equipped with an air filter unit 58 as shown best in FIGS. 4-5. This air filter unit 58 comprises a compact housing mounted preferably at a rear side of the upper support rim 40, and functions to filter ambient air drawn into the otherwise sealed reservoir 12 during normal water cooler operation. That is, the air filter unit 58 is mounted along and defines an air inflow path through which ambient air can be drawn via a plurality of small inflow ports 60 through a suitable filtration media contained within the compact unit housing, and further past the seal gasket 46 into an upper region of the reservoir interior above the level of water contained therein. Such air inflow to the reservoir 12 is required to accommodate air-water exchange between the reservoir 12 and the bottle interior, as water downflow from the bottle replenishes water dispensed from the reservoir via the faucet valves 26 or 27. The air filter unit 58 insures that ambient air drawn into the reservoir 12 is substantially free of airborne contaminants particularly such as dirt, grit and bacterial and other airborne micro-organisms. The specific construction of the air filter unit 58 may vary, with one preferred configuration being shown and described in U.S. Pat. No. 6,167,921 which is incorporated by reference herein, and wherein the air filter unit 58 further includes a float-actuated check valve to prevent reservoir over-filling as might otherwise occur with use of a bottle 22 having a small crack or leak formed therein.

In accordance with a primary aspect of the invention, the combined unit 10 further includes means for introducing a sanitizing agent into the otherwise sealed interior of the cooler reservoir 12 to maintain the reservoir internal water-contacted surfaces and the reservoir-contained water in a clean and sanitary state. In the preferred form, this agent-introducing means comprises a gas injection system for periodically introducing a sanitizing gas, such as ozone, into the cooler reservoir 12.

More particularly, the flow line 24 is carried by the adapter 11 to extend from a first end coupled to a sanitizing agent source 62 located outside the reservoir 12, to a second end carrying a porous delivery element 64 located inside the reservoir 12 preferably at a location near the bottom thereof. FIGS. 4-5 show this flow line 24 coupled through a rear side of the adapter support rim 40 at a position disposed alongside the air filter unit 58. FIG. 6 shows the flow line 24 extending from the sanitizing agent source 62, such as a small ozone generator which may be mounted at or onto a rear side of the cooler cabinet 28, and a one-way check valve 66 mounted alongside the air filter unit 58 at the rear side of the adapter support rim 40. From this check valve 66, a continuation segment of the flow line 24 extends radially inwardly and then downwardly between the sleeve and inner cup segments 42, 50 of the adapter 11, and further downwardly through an open gap 68 (FIG. 5) in the baffle plate 32 into the lower reservoir chamber 12b. At this location, a lower end of the flow line 24 is coupled via a short elbow segment 70 to the porous delivery element 64 such as a porous stone member located substantially at the bottom of the reservoir 12.

In use, the source 62 is activated periodically to deliver the sanitizing agent such as ozone gas through the flow line 24 into the otherwise sealed reservoir 12. Such gaseous agent is pumped under a sufficient elevated pressure for delivery through the porous element 64, which converts the gaseous stream into a myriad of small bubbles rising upwardly through the water contained within the reservoir 12. These sanitizing bubbles effectively scrub internal surfaces of the reservoir and other immersed structures therein (such as the baffle plate 32) to sanitize these structures as well as to sanitize the water then-contained within the reservoir. Importantly, excess pressurization of the reservoir interior is prevented by escape of the gaseous agent through the air filter unit 58. In this regard, a float-activated check valve incorporated into the air filter unit 58 will prevent water outflow from the reservoir 12, but will not preclude venting of the gaseous agent during a sanitizing cycle.

The sanitizing agent source 62 is preferably operated for a short cycle sufficient to sanitize the reservoir surface and the reservoir-contained water, such as a sanitizing cycle of about 10 minutes. This sanitizing cycle is repeated at regular intervals, such as one cycle per day. Moreover, this sanitizing cycle preferably occurs at a time when water dispensing from the cooler 14 is not required, such as in the middle of the night in a typical residential or business establishment water cooler installation.

A controller 72 is conveniently provided on the adapter 11 at a user-accessible position, and is designed for quick and easy user manipulation to regulate the specific time and duration of each sanitizing cycle. In the preferred form as shown, the controller 72 comprises a compact electronic unit positioned at a front side of the adapter support rim 40 for each user access and viewing. The illustrative controller 72 includes a digital display 74 in association with control buttons 76 used to set a specific time-of-day, such as 2:00 a.m., for initiating the sanitizing cycle, and to set a specific cycle duration, such as about 10 minutes.

The combined feed tube adapter and ozonator unit 10 of the present invention beneficially accommodates periodic sanitization of the reservoir 12 in a manner that is fully compatible with use of a feed tube adapter. In addition, the invention permits quick and easy retro-fit of bottled water coolers by simple interchange of an existing feed tube adapter with the combined unit 10 to provide the additional and desirable sanitizing function, and without requiring complicated retro-fit plumbing procedures.

A variety of modifications and improvements in and to the combined feed tube adapter and sanitizing unit 10 of the present invention will be apparent to those persons skilled in the art. Accordingly, no limitation on the invention is intended by way of the foregoing description and accompanying drawings, except as set forth in the appended claims.

What is claimed is:

1. In a feed tube adapter having an adapter body for mounting generally over an upwardly open water reservoir of a bottled water cooler, and an upstanding feed tube for engaging and opening a bottle cap carried on the neck of a water bottle installed onto the bottled water cooler in an inverted orientation, the improvement comprising:
a flow line extending through said adapter body and into the water reservoir, when said adapter body is mounted over the water reservoir of the bottled water cooler, said flow line accommodating passage therethrough of a sanitizing agent into sanitizing contact with water contained within the water reservoir.

2. The improvement of claim 1 wherein said flow line has a first end disposed outside the water reservoir, and a second end disposed generally within the water reservoir at a location near the bottom thereof, when said adapter body is mounted over the water reservoir, and further including a porous delivery element carried by said flow line second end.

3. The improvement of claim 2 further including a sanitizing agent source coupled to said flow line first end.

4. The improvement of claim 3 wherein said sanitizing agent source comprises an ozone generator.

5. The improvement of claim 3 wherein said porous delivery element is adapted to deliver the sanitizing agent to the water contained within the water reservoir in the form of a myriad of small gas bubbles.

6. The improvement of claim 2 further including a controller for variably selecting an operating cycle for said sanitizing agent source.

7. The improvement of claim 6 wherein said controller is mounted on the bottled water cooler in a user-accessible position.

8. The improvement of claim 1 further including means for venting gas from the water reservoir.

9. The improvement of claim 8 wherein said venting means comprises an air filter mounted on said adapter body, said air filter including a float for preventing water outflow therethrough.

10. In a feed tube adapter having an adapter body for mounting generally over an upwardly open water reservoir of a bottled water cooler, and an upstanding feed tube for engaging and opening a bottle cap carried on the neck of a water bottle installed onto the bottled water cooler in an inverted orientation, the improvement comprising:
a flow line extending through said adapter body and into the water reservoir, when said adapter body is mounted over the water reservoir of the bottled water cooler, said flow line accommodating passage therethrough of a sanitizing agent into sanitizing contact with water contained within the water reservoir, said flow line having a first end disposed outside the water reservoir, and a second end disposed generally within the water reservoir at a location near the bottom thereof, when said adapter body is mounted over the water reservoir;
a sanitizing agent source coupled to said flow line first end;
a porous delivery element carried by said flow line second end; and
vent means on said adapter body for preventing gas pressurization within the water reservoir.

11. The improvement of claim 10 wherein said sanitizing agent source comprises an ozone generator.

12. The improvement of claim 10 wherein said porous delivery element is adapted to deliver the sanitizing agent to the water contained within the water reservoir in the form of a myriad of small gas bubbles.

13. The improvement of claim 10 further including a controller for variably selecting an operating cycle for said sanitizing agent source.

14. The improvement of claim 13 wherein said controller is mounted on the bottled water cooler in a user-accessible position.

15. The improvement of claim 10 wherein said vent means comprises an air filter mounted on said adapter body, said air filter including a float for preventing water outflow therethrough.

16. A bottled water cooler, comprising:
an upwardly open water reservoir;
a feed tube adapter including an adapter body mounted generally over said water reservoir, and a feed tube upstanding from said adapter body for engaging and opening a bottle cap carried on the neck of a water bottle installed thereon in an inverted orientation to permit water downflow from the water bottle through said feed tube and into said water reservoir;
dispense means for dispensing water from said water reservoir; and
a sanitizer unit including a sanitizing agent source positioned outside said water reservoir, and a flow line extending from said sanitizing agent source and through said adapter body and into said water reservoir.

17. The bottled water cooler of claim 16 wherein said water reservoir is mounted within a cooler housing, and further wherein said sanitizing agent source comprises a sanitizing gas generator mounted on said cooler housing.

18. The bottled water cooler of claim 17 further including a controller mounted on said cooler housing in a user-accessible position for variably selecting an operating cycle for said sanitizing agent source.

19. The bottled water cooler of claim 16 further including a porous delivery element mounted on a downstream end of said flow line within said water reservoir generally at a lower end thereof, said porous delivery element being adapted to deliver the sanitizing agent to the water contained within the water reservoir in the form of a myriad of small gas bubbles.

20. The bottled water cooler of claim 16 further including an air filter mounted on said adapter body, said air filter preventing gas pressurization of said water reservoir and including a float for preventing water outflow therethrough.
